(19) United States
(12) Patent Application Publication
(10) Pub. No.: US 2010/0204460 A1
(43) Pub. Date: Aug. 12, 2010

(54) NATURAL BRASSINOSTEROIDS FOR USE FOR TREATING HYPERPROLIFERATION, TREATING PROLIFERATIVE DISEASES AND REDUCING ADVERSE EFFECTS OF STEROID DYSFUNCTION IN MAMMALS, PHARMACEUTICAL COMPOSITION AND ITS USE

(76) Inventors: Jana Oklestková, Olomouc (CZ); Lucie Hoffmannová, Pribor (CZ); Jana Steigerová, Olomouc (CZ); Ladislav Kohout, Praha (CZ); Zdenêk Kolár, Prostejov (CZ); Miroslav Strnad, Olomouc (CZ)

Correspondence Address: NOTARO, MICHALOS & ZACCARIA P.C., 100 DUTCH HILL ROAD, ORANGEBURG, NY, 10962, US

(21) Appl. No.: 12/679,793

(22) PCT Filed: Aug. 20, 2008
(86) PCT No.: PCT/CZ08/00097

§ 371 Date: Mar. 24, 2010

(30) Foreign Application Priority Data
Aug. 22, 2007 (CZ) PV 2007-571

(51) Int. Cl. C07J 17/00; C07D 313/10; C07J 9/00; C07J 71/00

(52) U.S. Cl. 536/5; 549/268; 552/541; 552/542; 540/76

(57) ABSTRACT

The present invention relates to natural brassinosteroids of general formula (I), wherein R is CH₂ or O—CH₂ group, R² is hydrogen or hydroxyl, R³ is hydroxyl, R²⁴ is alkyl or alkenyl, which are selected from the group consisting of methyl, ethyl, propyl, isopropyl, methylen, ethylen and propylen, and R²⁵ is alkyl selected from the group consisting of methyl and ethyl, and a pharmaceutically acceptable salt thereof, for use for treating hyperproliferation, treating proliferative diseases and reducing adverse effects of steroid dysfunction in mammals. The present invention also provides methods capable to arrest of the cell cycle by natural brassinosteroids resulting in apoptotic changes in cancer cells. More specifically, the present invention relates to use for treatment of the adverse effects of hyperproliferation on mammalian cells in vitro and in vivo, especially treatment of hyperproliferative diseases in mammals by administering compositions containing natural brassinosteroids. This invention also describes new use for treating consisting in a new therapeutic way for modifying cell viability of human breast and prostate cancer cells.

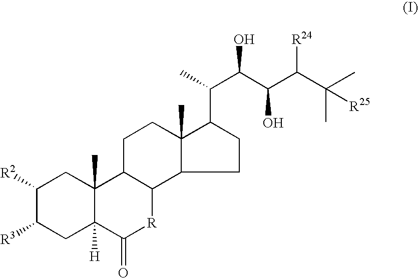

### FIELD OF INVENTION

This invention relates to natural brassinosteroids and their derivatives for use for the inhibition of hyperproliferation in mammalian cells, for treating proliferative diseases in mammals, and for regulation of the adverse effects of steroid disfunctions in mammalian cells and mammals. The present invention relates also to natural brassinosteroids and their derivatives for use in anticancer therapy.

### BACKGROUND OF THE INVENTION

Brassinosteroids are steroid plant hormones with important regulatory roles in various physiological processes, including growth, differentiation, root and stem elongation, disease resistance, stress tolerance and senescence (Bajguz et al., Phytochemistry 2003, 62, 1027-1046). This group of plant steroids includes more than 70 compounds distributed from lower to higher plants. Brassinosteroids have been detected and isolated from seeds, fruits, leaves, galls and pollen (Sasse J., In: Brassinosteroids: Steroidal Plant Hormones; Springer-Verlag, Tokyo, 1999; pp. 219-262; Khripach et al., A new class of Plant Hormones, Academic Press; San Diego, 1999, p. 456). Brassinosteroids are structurally very similar to animal steroid hormones. Like their animal counterparts, brassinosteroids regulate the expression of numerous genes, impact the activity of complex metabolic pathways, and contribute to the regulation of cell division and differentiation. They are also involved in regulating processes including photomorphogenesis and cell expansion in the presence of a potentially growth-limiting cell wall (Clouse, Current Biol. 2002, 12, 485-487). The high biological activity of brassinosteroids has attracted the attention of many specialists in the field of chemistry, biology, pharmacology and agriculture.

Brassinolide of the formula 1, the most biologically active brassinosteroid, was initially isolated from Brassica napus pollen (Grove et al., Nature 1979, 281, 216-217). Subsequently, brassinolide has been identified in all plant species examined up to now (Bajguz et al., Phytochemistry 2003, 62, 1027-1046; Zullo et al., Braz. J. Plant Physiol. 2002, 14, 143-181). Brassinolide 1 is present at very low levels in plant tissues (pmol/g fresh weight) and its synthesis is very difficult to carry out. Therefore, current research is focused mainly on development of equally effective and easily available compounds. Some of the synthetically prepared brassinosteroids were later found to occur in nature (Soeno et al., Biosc. Biotechnol. Biochem. 2000, 64, 702-709). An example of such a compound, synthetically more easily available in comparison to brassinolide, is its 24-epimer, 24-epibrassinolide (compound of formula 6; Back et al., J. Org. Chem. 1997, 62, 1179-1182). This brassinosteroid is therefore the most widely studied compound in this class and also is the best candidate for practical applications.

Brassinosteroids have been reported to have contradictory effects on cell division in different plant species and cultured cell lines. The effect of brassinosteroids on cell division has been shown to be mainly promotive. They substitute for the effects of cytokinins (both brassinosteroids and cytokinins induce cycD3 gene expression), and promote cell division during the early cell culture phases, suggesting that brassinosteroids are limiting factors in induction of cell cycle (Hu et al., Plant J. 2000, 24, 693-701, Miyazawa et al. J. Exp. Bot. 2003, 54, 2669-2678).

Some medical applications of brassinosteroids have also already been reported. Wachsman et al. (Antivir. Chem. Chemother. 2000, 11, 71-77; Chemother. 2002, 13, 61-66) reported that some natural brassinosteroids (28-homocastasterone of formula 9, 28-homobrassinolide of formula 2) and their synthetic analogues have in vitro antiviral activity against several pathogenic viruses, such as herpes simplex virus type 1 (HSV-1), arena viruses and measles virus (MV). Several of the brassinosteroid analogues have been shown to be 10- to 18-fold more active than ribavirin (used as the reference drug) for HSV-1 and arena viruses. However, further studies are needed to define the precise in vitro antiviral mechanism of these brassinosteroid analogues and to correlate molecular structure and bioactivity. There is also one report describing possible effects of 24-epibrassinolide on cultured mouse hybridoma cells. Typical effects of compound 6 are: (a) increase in the value of mitochondrial membrane potential, (b) decrease of intracellular antibody level, (c) increase in the fraction of the cells in G₀/G₁ phase, and (d) vice versa decrease of S-phase cells. Furthermore, the density of viable cells was significantly higher at 24-epibrassinolide concentrations of 10⁻¹³ mol/L and 10⁻¹² mol/L (Fran\check{e}k et al., Collect. Czech Chem. Commun. 2003, 68, 2190-2200).

We have found that a series of natural brassinosteroids are effective in growth inhibition of many different cancer cell lines at micromolar concentrations despite of their minimum effects on normal cells. Their cytotoxic activity could be, at least partially, related to interactions with steroid receptors. Brassinosteroids are a quite new group of compounds, not yet completely understood as far as the mechanism of their action on a molecular level is concerned. We have shown that brassinosteroid application induces cytotoxicity and growth inhibition of breast and prostate carcinoma cells. Hence, they can be used as antimitotic and apoptotic drugs, particularly as anticancer drugs.

It is a subject of this invention to provide anticancer compounds having high selectivity and efficiency index, i.e. that they are less toxic and yet more efficacious than analogues known heretofore.

### SUMMARY OF THE INVENTION

The object of this invention are natural brassinosteroids of general formula I

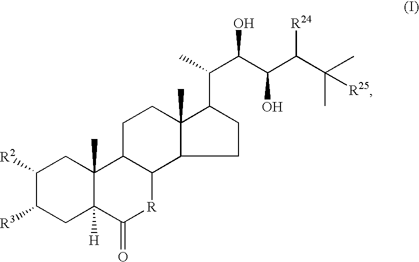

wherein
R is CH₂ or O—CH₂ group,
R² is hydrogen or hydroxyl,
R³ is hydroxyl,
R²⁴ is alkyl or alkenyl, which are selected from the group consisting of methyl, ethyl, propyl, isopropyl, methylen, ethylen and propylen, and
R²⁵ is alkyl selected from the group consisting of methyl and ethyl,
   and pharmaceutically acceptable salts thereof.

The present invention relates also to use of compounds of general formula I for the treatment of hyperproliferation and of proliferative diseases and for reducing adverse effects of steroid dysfunction in mammals. It also relates to pharmaceutical composition containing such compounds and use of this pharmaceutical composition.

The present invention relates to natural brassinosteroids of general formula I for use for the inhibition of cell proliferation and for the induction of apoptosis.

In another embodiment, this invention relates to natural brassinosteroids of general formula I for use for the inhibition of cell proliferation and for inducing apoptosis in mammalian cells, comprising administration of a therapeutically effective amount of the brassinosteroids of general formula I or their pharmaceutically-acceptable salts. Natural brassinosteroids are useful especially for treating disorders, some of them involving cell proliferation, and including cancer, Alzheimer disease, Huntington disease, steroid-induced osteoporesis, sexual differentiation disorders, hyperadrenocorticism associated with sex steroid excess, androgen insensitivity syndrome, glucocorticoid insensitive asthma, steroid-induced cataracta, and deficiency of P450 oxidoreductase.

A further subject of this invention is the use of natural brassinosteroids of general formula I as growth regulator in animal and human tissue cultures for regulation of proliferation and morphogenesis.

This invention also concerns natural brassinosteroid derivatives according to claim 1 of the general formula I for use as drugs.

This invention also relates to pharmaceutical compositions comprising natural brassinosteroids and/or their substituted analogues, and a pharmaceutically acceptable carrier(s).

In yet another embodiment, this invention relates to a pharmaceutical composition(s) comprising the composition in an admixture with one or more pharmaceutical excipients.

The present invention relates also to brassinosteroid derivatives which may be used in compositions in the form of free compounds of the above given general formulae I or as pharmaceutically acceptable salts thereof. Pharmaceutically acceptable salts may include, for example, those with alkali metals, ammonium, or amines. They may also be in the form of addition salts with acid. The derivatives or their salts may be in the form of a racemate mixture or optically active isomers.

Thus, the present invention also relates to brassinosteroids or their derivatives for use for treating hyperproliferative diseases in mammalian cells, the said method comprising an application of an effective amount of a natural brassinosteroid derivative(s) to the mammalian cells in need of such treatment.

### DETAILED DESCRIPTION

The present invention relates to natural brassinosteroid derivatives of the general formula I

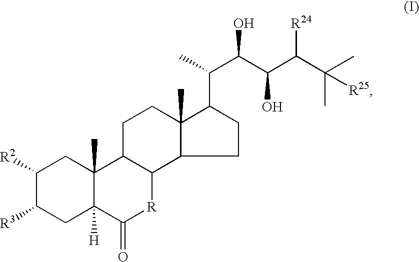

wherein
R is CH₂ or O—CH₂ group,
R² is hydrogen or hydroxyl,
R³ is hydroxyl,
R²⁴ is alkyl or alkenyl, which are selected from the group consisting of methyl, ethyl, propyl, isopropyl, methylen, ethylen and propylen, and
R²⁵ is alkyl selected from the group consisting of methyl and ethyl,
   and pharmaceutically acceptable salts thereof.

These natural brassinosteroid derivatives have been found to have anticancer properties when contacted with mammalian cells, including human cells.

As used herein, and unless modified by the immediate context the generic substituent groups have meanings identical with the definitions of the corresponding groups as defined in this description, wherein

alkyl denotes
C₁-C₃ branched or unbranched alkyl, preferentially selected from the group consisting of methyl, ethyl, propyl, isopropyl,
   alkenyl denotes
C₁-C₃ branched or unbranched alkenyl, preferentially selected from the group consisting of methylen, ethylen, propylen, isopropylen,
   hydrogen denotes H,
   hydroxyl denotes the group —OH,

In a preferred embodiment the invention relates to natural brassinosteroids of general formula I, especially including compounds of the following formulas 1 to 47:

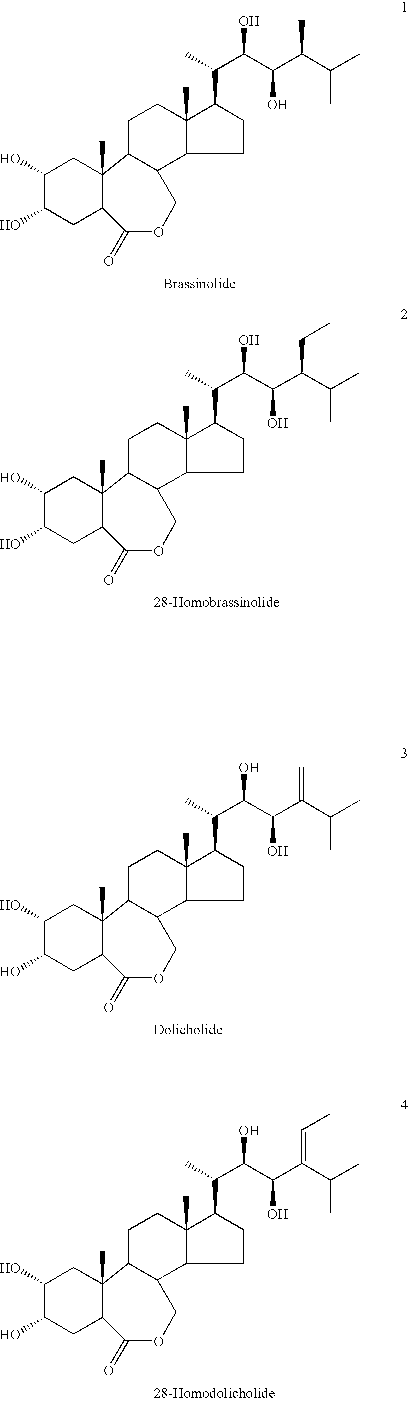

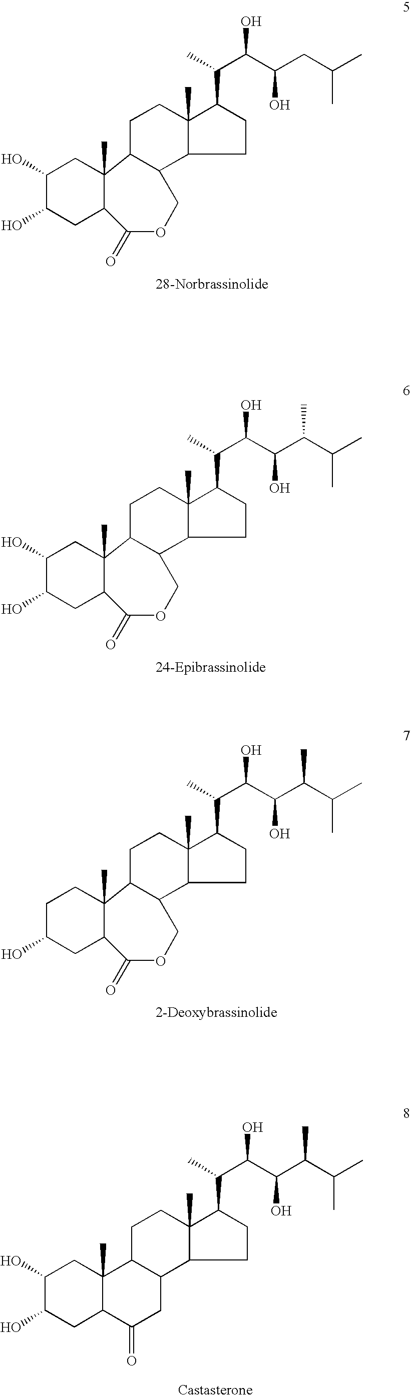

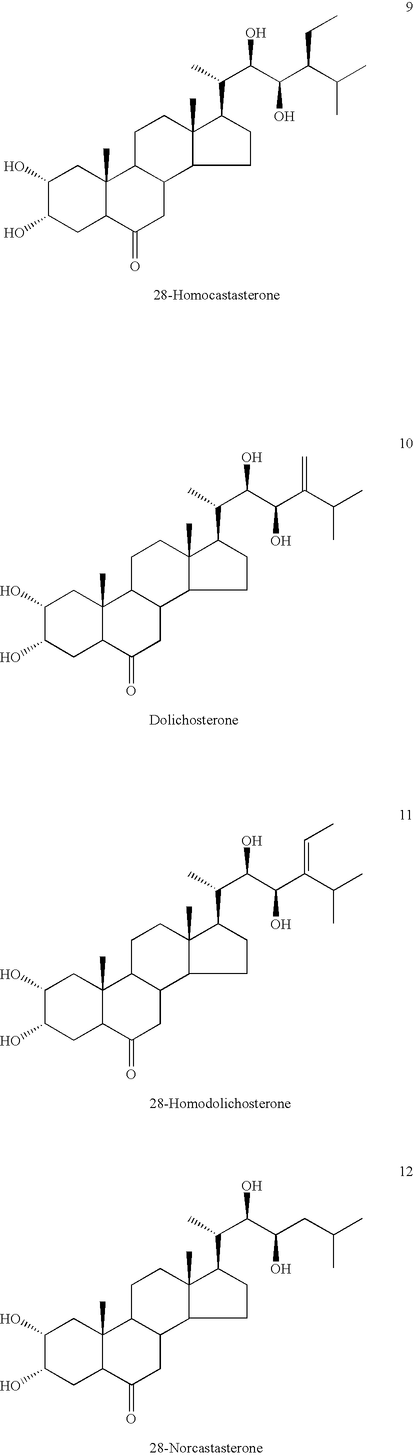

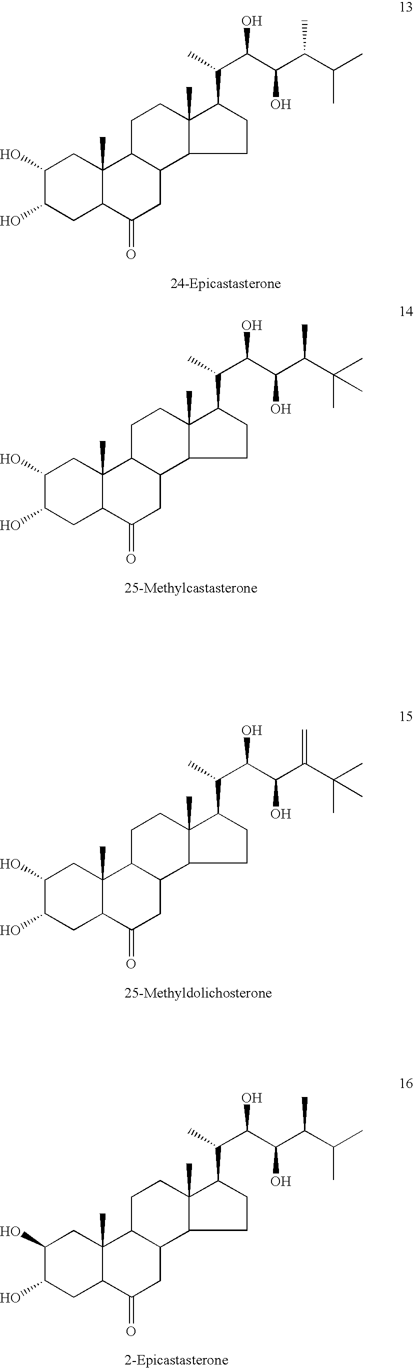

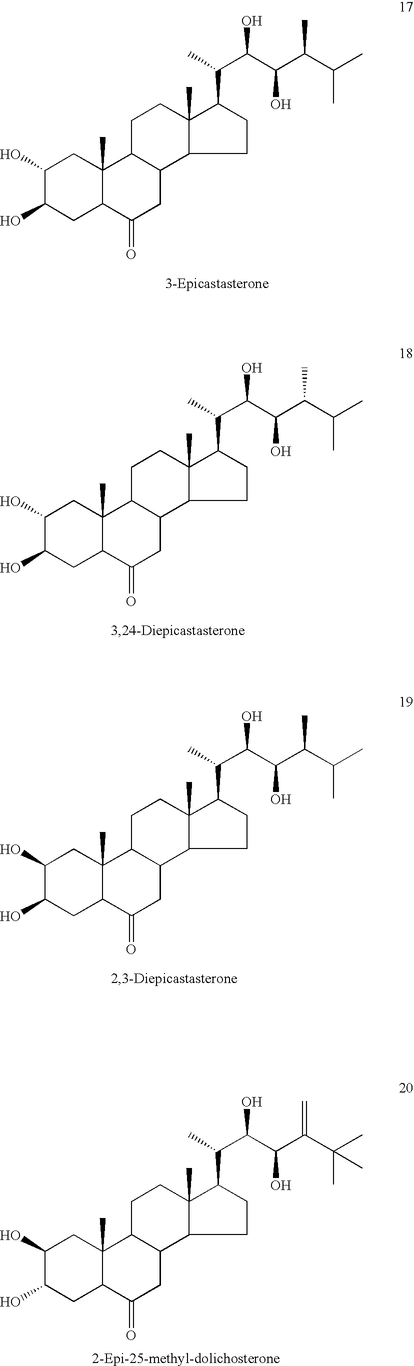

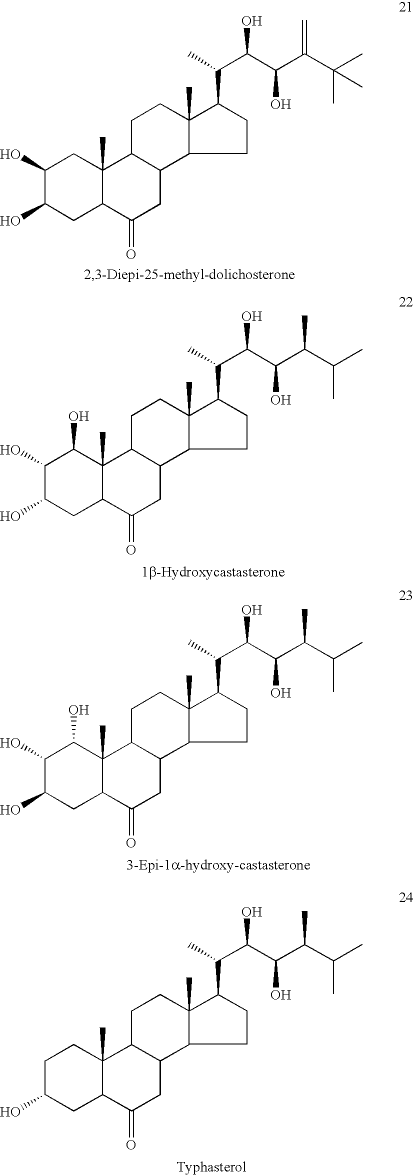

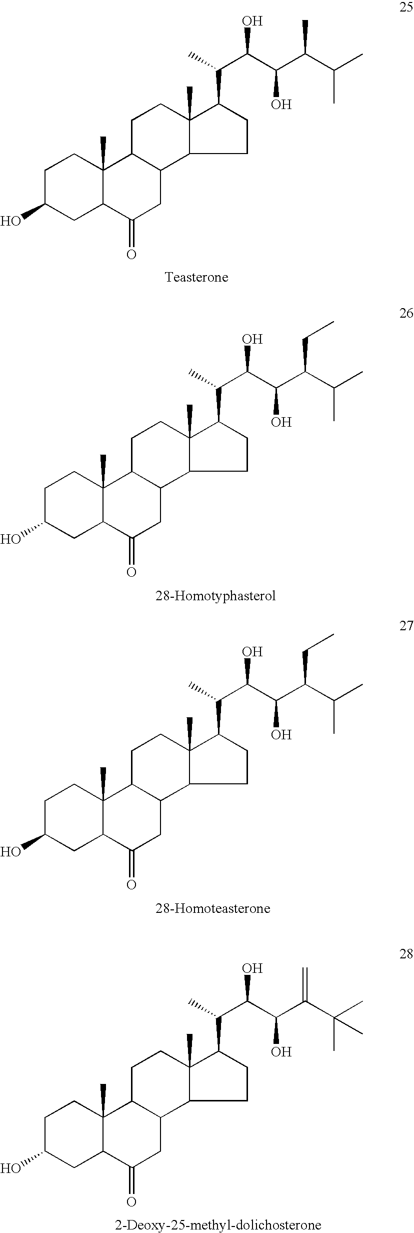

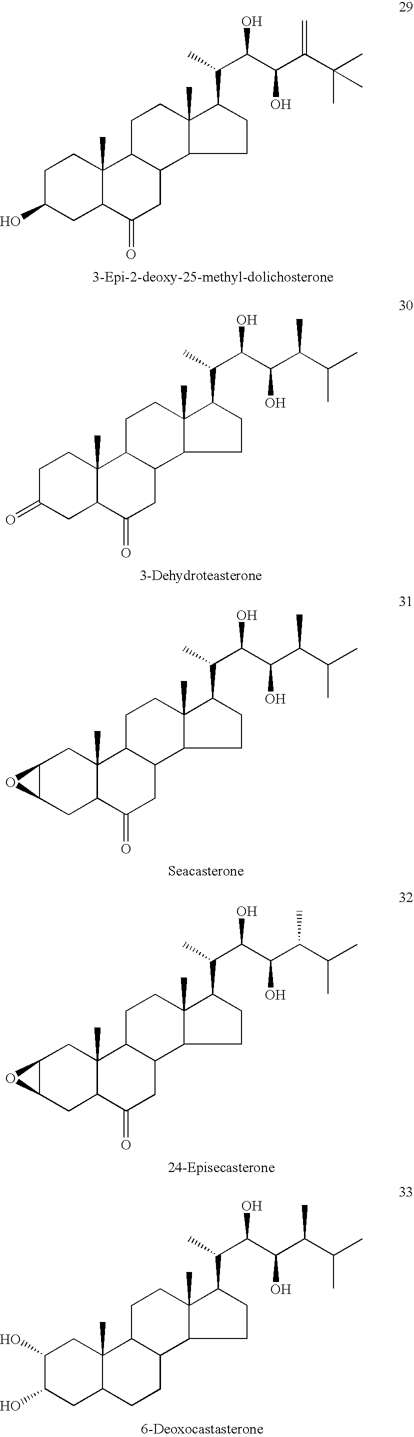

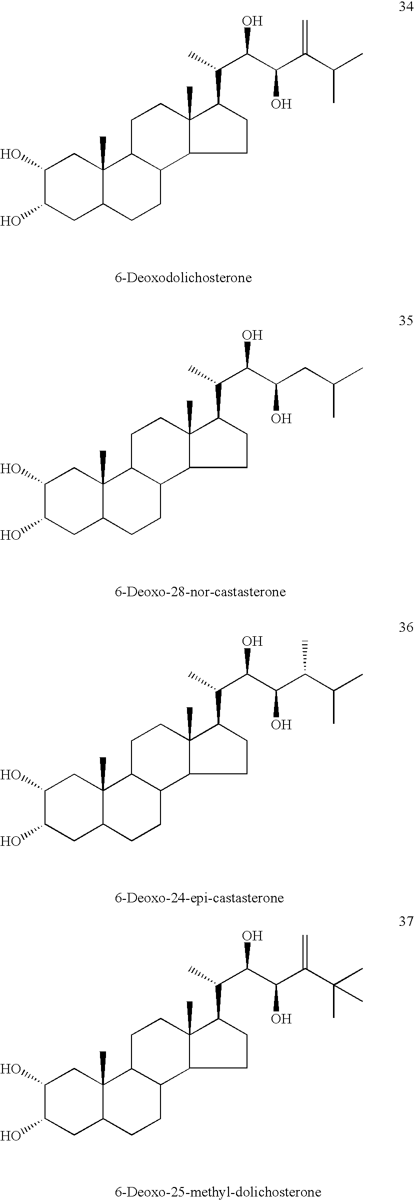

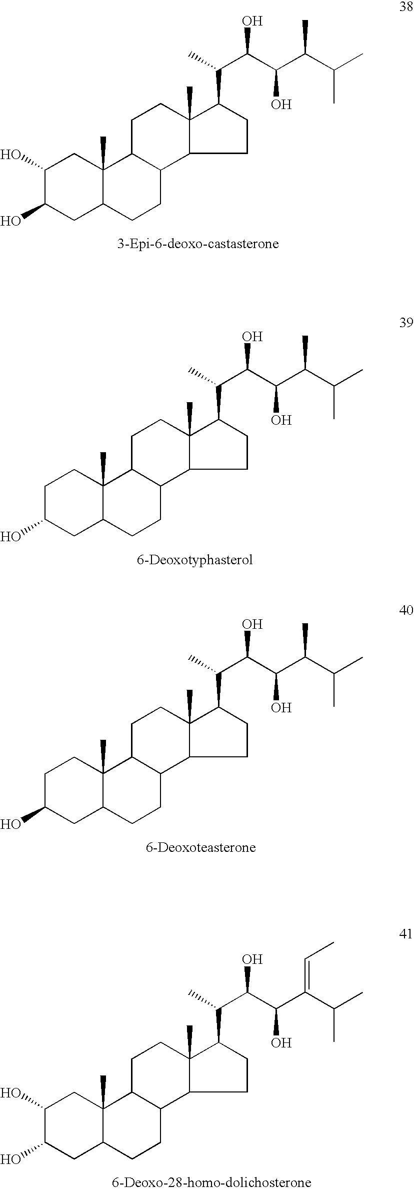

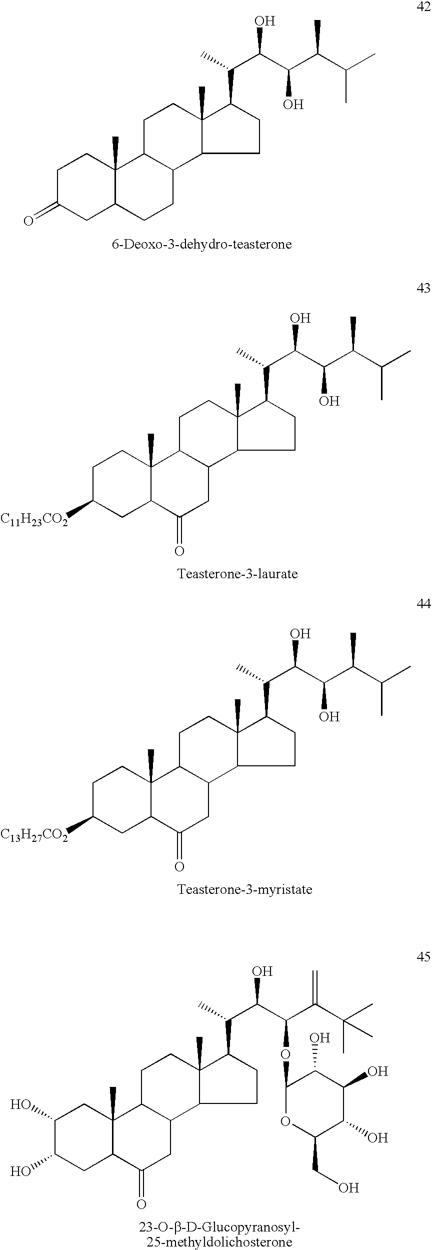

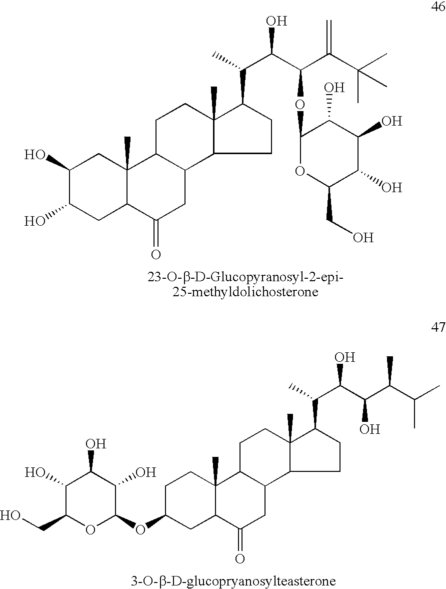

and the pharmaceutically acceptable acid salts thereof.

The following derivatives are particularly preferred: castasterone, 28-homocastasterone, 24-epibrassinolide, dolichosterone, 2-deoxycastasterone, typhasterol, teasterone, 3-oxoteasterone, cathasterone, 6-deoxotyphasterol, 3-dehydro-6-deoxoteasterone, homotyphasterol, homoteasterone, homodolichosterone, 25-methylcastasterone, 25-methyldolichosterone, 2-deoxy-25-methyldolichosterone, 3-epi-2-deoxy-25-methyldolichosterone.

### Therapeutic Administration

Suitable routes for administration include oral, rectal, topical (including ocular, buccal and sublingual), vaginal and parenteral (including subcutaneous, intramuscular, intravitreous, intravenous, intradermal, intrathecal and epidural) way. The preferred route of administration will depend upon the condition of the patient, the toxicity of the compound and the type and site of infection, among other considerations known to the clinician.

The therapeutic composition comprise about 1% to about 95% of the active ingredient, single-dose forms of administration preferably comprising about 20% to about 90% of the active ingredient and administration forms, which are not single-dose preferably comprising about 5% to about 20% of the active ingredient. Single dose forms may be, for example, coated tablets, tablets, ampoules, vials, suppositories or capsules. Other forms of administration are, for example, ointments, creams, pastes, foams, tinctures, lipsticks, drops, sprays, dispersions and the like. Examples are capsules containing from about 0.05 g to about 1.0 g of the active ingredient.

The pharmaceutical compositions of the present invention are prepared in a manner known per se, for example by means of conventional mixing, granulating, coating, dissolving or lyophilizing processes.

Preferably, solutions of the active ingredient, and in addition also suspensions or dispersions, especially isotonic aqueous solutions, dispersions or suspensions, are used, if being possible for these to be prepared before use, for example in the case of lyophilised compositions which comprise the active substance by itself or together with a carrier, for example mannitol. The pharmaceutical compositions can be sterilised and/or comprise excipients, for example preservatives, stabilisers, wetting agents and/or emulsifiers, solubilizing agents, salts for regulating the osmotic pressure and/or buffers, and they are prepared in a manner known per se, for example by means of conventional dissolving or lyophilising processes. The solutions or suspensions mentioned can comprise viscosity-increasing substances, such as sodium carboxymethylcellulose, dextran, polyvinylpyrrolidone or gelatine.

Suspensions in oil comprise, as the oily component, the vegetable, synthetic or semi-synthetic oils customary for injection purposes. Oils which may be mentioned are, in particular, liquid fatty acid esters which contain, as the acid component, a long-chain fatty acid having 8-22, in particular 12-22, carbon atoms, for example lauric acid, tridecylic acid, myristic acid, pentadecylic acid, palmitic acid, margaric acid, stearic acid, arachidonic acid, behenic acid or corresponding unsaturated acids, for example oleic acid, elaidic acid, euric acid, brasidic acid or linoleic acid, if appropriate with the addition of antioxidants, for example vitamin E, β-carotene or 3,5-di-tert-butyl-4-hydroxytoluene. The alcohol component of these fatty acid esters has not more than 6 carbon atoms and is mono- or polyhydric, for example mono-, di- or trihydric alcohol, for example methanol, ethanol, propanol, butanol, or pentanol, or isomers thereof, but in particular glycol and glycerol.

Fatty acid esters are, for example: ethyl oleate, isopropyl myristate, isopropyl palmitate, “Labrafil M 2375” (polyoxyethylene glycerol trioleate from Gattefoseé, Paris), “Labrafil M 1944 CS” (unsaturated polyglycolated glycerides prepared by an alcoholysis of apricot kernel oil and made up of glycerides and polyethylene glycol esters; from Gattefoseé, Paris), “Labrasol” (saturated polyglycolated glycerides prepared by an alcoholysis of TCM and made up of glycerides and polyethylene glycol esters; from Gattefoseé, Paris) and/or “Miglyol 812” (triglyceride of saturated fatty acids of chain length C₈ to C₁₂ from Hüls AG, Germany), and in particular vegetable oils, such as cottonseed oil, almond oil, olive oil, castor oil, sesame oil, soybean oil and, in particular, groundnut oil.

The preparation of the injection compositions is carried out in the customary manner under sterile conditions, as are bottling, for example into ampoules or vials, and closing of the containers.

For example, pharmaceutical compositions for oral use can be obtained by combining the active ingredient with one or more solid carriers, if appropriate granulating the resulting mixture, and, if desired, processing the mixture or granules to tablets or coated tablet cores, if appropriate by addition of additional excipients.

Suitable carriers are, in particular, fillers, such as sugars, for example lactose, sucrose, mannitol or sorbitol, cellulose preparations and/or calcium phosphates, for example tricalcium diphosphate, or calcium hydrogen phosphate, and furthermore binders, such as starches, for example maize, wheat, rice or potato starch, methylcellulose, hydroxypropylmethylcellulose, sodium carboxymethylcellulose and/or polyvinylpyrrolidine, and/or, if desired, desintegrators, such as the above mentioned starches, and furthermore carboxymethyl-starch, cross-linked polyvinylpyrrolidone, alginic acid or a salt thereof, such as sodium alginate. Additional excipients are, in particular, flow regulators and lubricants, for example salicylic acid, talc, stearic acid or salts thereof, such as magnesium stearate or calcium stearate, and/or polyethylene glycol, or derivatives thereof.

Coated tablet cores can be provided with suitable coatings which, if appropriate, are resistant to gastric juice, the coatings used being, inter alia, concentrated sugar solutions, which, if appropriate, comprise gum arabic, talc, polyvinylpyrrolidine, polyethylene glycol and/or titanium dioxide, coating solutions in suitable organic solvents or solvent mixtures or, for the preparation of coatings which are resistant to gastric juice, solutions of suitable cellulose preparations, such as acetylcellulose phthalate or hydroxypropylmethylcellulose phthalate. Dyes or pigments can be admixed to the tablets or coated tablet coatings, for example for identification or characterisation of different doses of active ingredient.

Pharmaceutical compositions, which can be used orally, are also hard capsules of gelatine and soft, closed capsules of gelatine and a plasticiser, such as glycerol or sorbitol. The hard capsules can contain the active ingredient in the form of granules, mixed for example with fillers, such as maize starch, binders and/or lubricants, such as talc or magnesium stearate, and stabilisers if appropriate. In soft capsules, the active ingredient is preferably dissolved or suspended in suitable liquid excipients, such as greasy oils, paraffin oil or liquid polyethylene glycol or fatty acid esters of ethylene glycol or propylene glycol, it being likewise possible to add stabilisers and detergents, for example of the polyethylene sorbitan fatty acid ester type.

Other oral forms of administration are, for example, syrups prepared in the customary manner, which comprise the active ingredient, for example, in suspended form and in a concentration of about 5% to 20%, preferably about 10% or in a similar concentration which results in a suitable individual dose, for example, when 5 or 10 ml are measured out. Other forms are, for example, also pulverulent or liquid concentrates for preparing of shakes, for example in milk. Such concentrates can also be packed in unit dose quantities.

Pharmaceutical compositions, which can be used rectally, are, for example, suppositories that comprise a combination of the active ingredient with a suppository base. Suitable suppository bases are, for example, naturally occurring or synthetic triglycerides, paraffin hydrocarbons, polyethylene glycols or higher alkanols.

Compositions which are suitable for parental administration are aqueous solutions of an active ingredient in water-soluble form, for example of water-soluble salt, or aqueous injection suspensions, which comprise viscosity-increasing substances, for example sodium carboxymethylcellulose, sorbitol and/or dextran, and, if appropriate, stabilizers. The active ingredient can also be present here in the form of a lyophilisate, if appropriate, together with excipients, and be dissolved before parenteral administration by addition of suitable solvents. Solutions such as are used, for example, for parental administration can also be used as infusion solutions. Preferred preservatives are, for example, antioxidants, such as ascorbic acid, or microbicides, such as sorbic or benzoic acid.

Ointments are oil-in-water emulsions which comprise not more than 70%, preferably 20-50% of water or aqueous phase. The fatty phase consists, in particular, hydrocarbons, for example vaseline, paraffin oil or hard paraffins, which preferably comprise suitable hydroxy compounds, such as fatty alcohols or esters thereof, for example cetyl alcohol, or wool wax alcohols, such as wool wax, to improve the water-binding capacity. Emulsifiers are corresponding lipophilic substances, such as sorbitan fatty acid esters (Spans), for example sorbitan oleate and/or sorbitan isostearate. Additives to the aqueous phase are, for example, humectants, such as polyalcohols, for example glycerol, propylene glycol, sorbitol and/or polyethylene glycol, or preservatives and odoriferous substances.

Fatty ointments are anhydrous and comprise, as the base, in particular, hydrocarbons, for example paraffin, vaseline or paraffin oil, and furthermore naturally occurring or semi-synthetic fats, for example hydrogenated coconut-fatty acid triglycerides, or, preferably, hydrogenated oils, for example hydrogenated groundnut or castor oil, and furthermore fatty acid partial esters of glycerol, for example glycerol mono- and/or distearate. They also contain emulsifiers and/or additives mentioned in connection with the ointments that increase uptake of water.

Creams are oil-in-water emulsions, which comprise more than 50% of water. Oily bases used are, in particular, fatty alcohols, for example lauryl, cetyl or stearyl alcohols, fatty acids, for example palmitic or stearic acid, liquid to solid waxes, for example isopropyl myristate, wool wax or beeswax, and/or hydrocarbons, for example vaseline (petrolatum) or paraffin oil. Emulsifiers are surface-active substances with predominantly hydrophilic properties, such as corresponding non-ionic emulsifiers, for example fatty acid esters of polyalcohols or ethyleneoxy adducts thereof, such as polyglyceric acid fatty acid esters or polyethylene sorbitan fatty esters (Tween), and furthermore polyoxyethylene fatty alcohol ethers or polyoxyethylene fatty acid esters, or corresponding ionic emulsifiers, such as alkali metal salts of fatty alcohol sulphates, for example sodium lauryl sulphate, sodium cetyl sulphate or sodium stearyl sulphate, which are usually used in the presence of fatty alcohols, for example cetyl stearyl alcohol or stearyl alcohol. Additives to the aqueous phase are, inter alia, agents which prevent the creams from drying out, for example polyalcohols, such as glycerol, sorbitol, propylene glycol and/or polyethylene glycols, and furthermore preservatives and odoriferous substances.

Pastes are creams and ointments having secretion-absorbing powder constituents, such as metal oxides, for example titanium oxide or zinc oxide, and furthermore talc and/or aluminium silicates, which have the task of binding the moisture or secretions present.

Foams are administered from pressurised containers and they are liquid oil-in-water emulsions present in aerosol foam. Gases halogenated hydrocarbons, such as polyhalogenated alkanes, for example dichlorofluoromethane and dichlorotetrafluoroethane, or, preferably, non-halogenated gaseous hydrocarbons air, N₂O and/or carbon dioxide are used as propellant gases. The oily phases used are, inter alia, those mentioned above for ointments and creams, and the additives mentioned there are likewise used.

Tinctures and solutions usually comprise an aqueous-ethanolic base to which, humectants for reducing evaporation, such as polyalcohols, for example glycerol, glycols and/or polyethylene glycol, and re-oiling substances, such as fatty acid esters with lower polyethylene glycols, i.e. lipophilic substances soluble in the aqueous mixture to substitute the fatty substances removed from the skin with ethanol, and, if necessary, other excipients and additives, are admixed.

The present invention further provides veterinary compositions comprising at least one active ingredient as above defined together with a veterinary carrier therefor. Veterinary carriers are materials for administering the composition and may be solid, liquid or gaseous materials, which are inert or acceptable in the veterinary art and are compatible with the active ingredient. These veterinary compositions may be administered orally, parenterally or by any other desired route.

The invention also relates to a process or method for treatment of the disease states mentioned above. The compounds can be administered prophylactically or therapeutically as such or in the form of pharmaceutical compositions, preferably in an amount, which is effective against the diseases mentioned. With a warm-blooded animal, for example a human, requiring such treatment, the compounds are used, in particular, in the form of pharmaceutical composition. A daily dose of about 0.1 to about 50 g, preferably 0.5 g to about 10 g, of a compound of the present invention is administered here for a body weight of about 70 kg.

### BRIEF DESCRIPTION OF DRAWINGS

FIG. 1: The inhibitory effects of the 28-homocastasterone (28-homo-CS; 9; A) and 24-epibrassinolide (24-epiBL; 6; B) on cell viability in RPMI-8226 and CEM cancer cell lines. The data represent the means of three experiments±SD.

FIG. 2: Effect of 28-homoCS (9; A) and 24-epiBL (6; B) on cell viability in MCF-7 cell line assessed by MTT cell viability test (the cell viability of control cells was regarded as 100%). The data represent the means±SD of three independent experiments done in triplicate. * denotes the value that is significantly different from control value at p<0.05.

FIG. 3: Cell cycle analysis of MDA-MB-468 cell line by flow cytometry: A) untreated control, B) cells treated with 28-homoCS, and 24-epiBL treated cells. The M₁ region represents cells in the G₁, S, and G₂/M phases of cell cycle, and the M₂ region represents apoptotic cells with a reduced DNA content (subG₁ farction of cell cycle). Histograms of the treated cells were compared with control untreated cells. Horizontal and vertical axes indicate relative nuclear DNA content and number of cells, respectively.

FIG. 4: Western blot analysis of cell cycle related proteins (p53, MDM-2, p16, p21, p27, cyclin D₁, pRb-P) in breast (A) and prostate (B) cancer lines. The protein expressions of cells treated with 28-homocastasterone (9) (28-homoCS 6; 28-homoCS 12; 28-homoCS 24) and 24-epibrassinolide (6) (24-epiBL 6; 24-epiBL 12; 24-epiBL 24) for 6, 12 and 24 h were compared with the protein expression of control, untreated cells (C 6—control, 6 h; C 12—control, 12 h; C 24—control, 24 h). The expression of mcm-7 or α-tubulin was used as protein loading marker.

FIG. 5: Apoptotic cells identified by TUNEL assay in LNCaP prostate cancer cells treated with 28-homoCS or 24-epiBL in IC₅₀ concentration for 24 h compared to control, untreated cells.

FIG. 6: Western blot analysis of apoptosis related proteins (Bcl-2, Bcl-X_{L}, Bax, Bid, caspase-3) in breast (A) and prostate (B) cancer cells. The protein expressions of cells treated with 28-homocastasterone (9) (28-homoCS 6; 28-homoCS 12; 28-homoCS 24) and 24-epibrassinolide (6) (24-epiBL 6; 24-epiBL 12; 24-epiBL 24) in IC₅₀ concentrations for 6, 12 and 24 h were compared with the protein expression of control, untreated cells (C 6—control, 6 h; C 12—control, 12 h; C 24—control, 24 h). The expression of mcm-7 or α-tubulin was used as a protein loading marker.

The following examples serve to illustrate the invention without any limiting the scope thereof.

### EXAMPLES

### General Procedure

Stock solutions (10 μmol/L) were prepared by dissolving relevant quantity of substance in DMSO cultivation media (DMEM, RPMI 1640, F-12 medium), fetal bovine serum (FBS), L-glutamine, penicillin, and streptomycin were purchased from Sigma (MO, USA). 3-(4,5-Dimethylthiazol-2-yl)-2,5-diphenyltetrazolium bromide (MTT) was from Serva Electrophoresis (Heidelberg, Germany). Monoclonal mouse anti-bromodeoxyuridine (BrdU) intibody was obtained from DakoCytomation (Glostrup, Denmark). Calcein AM was obtained from PAA Laboratories GmbH (Pasching, Austria). The screening cell lines (T-lymphoblastic leukaemia cell line CEM; breast carcinoma cell line MCF-7 (estrogen-sensitive), breast adenocarcinoma cell line MDA-MB 468 (cell lines LNCaP (androgen-sensitive), prostate carcinoma cell line DU-145 (androgen-sensitive), lung carcinoma cell line A-549, chronic myelogenous leukemia cell line K562, multiple myeloma cell line RPMI 8226, cervical carcinoma cell line HeLa, malignant human melanoma cell line G361, osteosarcoma cell line HOS, and normal human fibroblasts (BJ) were obtained from the American Type Culture Collection (Manassas, Va., USA). MCF-7 cells were cultured in F-12 medium (Sigma, MO, USA). LNCaP cells were cultured in RPMI 1640 medium (GIBCO, USA). All other cells were cultured in DMEM medium (Sigma, MO, USA). All used media were supplemented with 10% heat-inactivated fetal bovine serum, 2 mmol/L L-glutamine, 1% penicillin/streptomycin. The cell lines were maintained under standard cell culture conditions at 37° C. and 5% CO₂ in a humid environment. Cells were subcultured twice or three times a week using the standard trypsinization procedure (Sigma, USA).

### Example 1

### Testing of In Vitro Cytotoxicity

The cell suspension of approximate density of 1.25×10⁵ cells/mL was redistributed into 96-well microtitre plates and after 12 h of stabilization the tested Brassinosteroids (BRs) were added in different concentrations. BRs were dissolved in DMSO. Control cultures were treated with DMSO alone. The final concentration of DMSO in the reaction mixture never exceeded 1%. Tested compounds in given concentrations were added at time zero in 20 μL aliquots to the microtiter plate wells. Usually, each test compound was evaluated at six 4-fold dilutions. In routine testing, the highest well concentration was 50 μmol, but it can be the matter of change dependent on the agent. Cultivation proceeds 96 h at 37° C., the cells were incubated with Calcein AM solution (Molecular Probes) for 1 h. Fluorescence (OD) of viable cells was quantified with Fluoroscan Ascent (Microsystems). The cell survival (IC₅₀) was calculated using the following equation: IC₅₀=(OD_{drug exposed well}/mean OD_{control wells})×100%. Each compound was tested in triplicates and the test was repeated at least 3 times. The IC₅₀ value, corresponding to drug concentration lethal to 50% of the tumor cells, was calculated from the obtained dose response curves.

We investigated effects of brassinosteroid on viability of normal and cancer cell lines of different histopathological origin. To evaluate the cytotoxic properties of several BRs and some related steroids, we used T-lymphoblastic leukaemia cell line CEM, breast carcinoma cell line MCF-7, lung carcinoma cell line A-549, chronic myeloid leukaemia cell line K562, multiple myeloma cell line RPMI 8226, cervical carcinoma cell line HeLa, malignant melanoma cell line G361, osteosarcoma cell line HOS, and normal human fibroblasts BJ. The cells were exposed to six 4-fold dilutions of each drug for 72 h to determinate number of surviving cells. The IC₅₀ values obtained from Calcein AM cytotoxicity assay are presented in Table 1.

TABLE 1
IC₅₀ (μmol/L) assessed by Calcein AM assay of surviving cells.

| | Compound (IC₅₀; | Cell line | | | |
|---|---|---|---|---|---|
| Brassinosteroid No. | μmol/L) | CEM | RPMI 8226 | G 361 | BJ |
| | Cholesterol | >50 | 38 ± 2.9 | >50 | >50 |
| | β-ecdyson | >50 | >50 | >50 | >50 |
| | 5α-cholestane | >50 | >50 | >50 | >50 |
| | Brassicasterol | >50 | >50 | >50 | >50 |
| | Stigmasterol | >50 | 32 ± 0.6 | >50 | >50 |
| | Sitosterol | >50 | 32 ± 1.6 | >50 | >50 |
| 1 | Brassinolide | >50 | >50 | >50 | >50 |
| 2 | 28-Homobrassinolide | 48 ± 1.3 | >50 | >50 | >50 |
| | 22S,23S-28- | 35 ± 2.2 | 31 ± 7.3 | >50 | >50 |
| | Homobrassinolide | | | | |
| 6 | 24-Epibrassinolide | 44 ± 2.2 | >50 | >50 | >50 |
| | 22S,23S-24- | >50 | >50 | >50 | >50 |
| | Epibrassinolide | | | | |
| 8 | Castasterone | 16 ± 5.3 | 33 ± 1.3 | >50 | >50 |
| 9 | 28-Homocastasterone | 13 ± 2.8 | 26 ± 1.4 | >50 | >50 |
| | 22S,23S-28- | 24 ± 1.5 | 25 ± 4.7 | 45 ± 2.2 | >50 |
| | Homocastasterone | | | | |
| 16 | 24-Epicastasterone | >50 | >50 | >50 | >50 |
| | 22S,23S-24- | 49 ± 1.8 | >50 | >50 | >50 |
| | Epicastasterone | | | | |

The results are means ± SD of three independent experiments performed in triplicate.

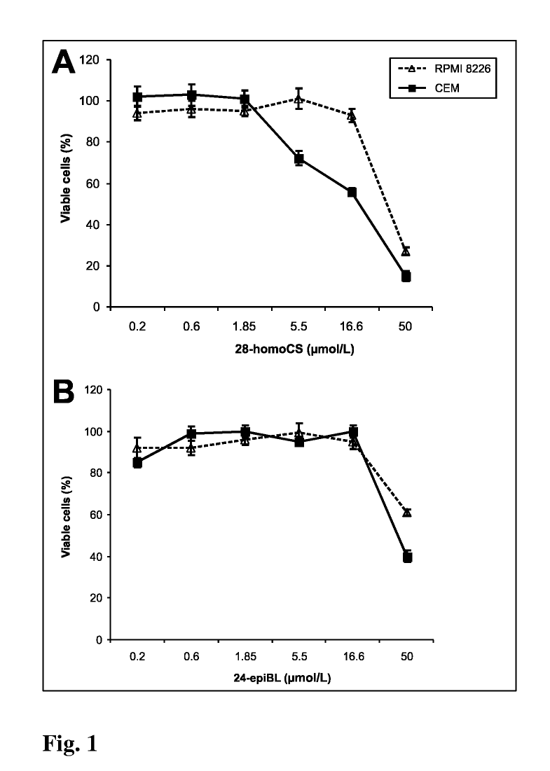

The treatment with brassinosteroids 9 and 6 resulted in a potent and dose dependent decrease in the viability on CEM and RPMI 8226 cells albeit at different levels (FIG. 1). Brassinosteroid 9 was the most potent compound on CEM cells (IC₅₀:13 μmol/L) while its 22S,23S-isomer was the most effective on RPMI 8226 (IC₅₀ 25 μmol/L). In addition to 9, the high cytotoxicity was also observed after application of castasterone and its artificial SS-homologue. The brassinolide, which is usually the most active compound in plant bioassays, is however inactive or exhibited almost zero cytotoxic activity, which artificial 22S,23S-28-homobrassinolide being the most effective (IC₅₀ 31-35 μmol/L). Almost zero activity was found towards any of the non-brassinosteroid plant sterols like cholesterol, stigmasterol, brassicosterol, 5α-cholestane, β-ecdysone, β-sitosterol and related compounds even when tested in amounts up to 50 μmol/L per assay. Some of the steroids, such as cholesterol, stigmasterol, and β-sitosterol showed only minimal anticancer activity (IC₅₀>30 μmol/L) on RPMI 8226 cell line. All tested compounds showed no cytotoxic effect on K 562, A 549, HeLa, and HOS cancer cell lines. A striking observation from this data was that on the BJ human fibroblasts, the brassinosteroid-mediated loss of viability was not observed. These results suggest a different response of steroids to cancer as compared to normal cells. At present, only few natural agents are known to posses the potential ability for selective/preferential elimination of cancer cells without affecting growth of normal cells. This study also provides the first evidence of brassinosteroids as anticancer compounds with antiproliferative properties. Up to now only a significant positive effect of 6 on the growth of mouse hybridoma cells in a concentration range of 10⁻¹²-10⁻¹³ mol/L has been shown.

To conclude the most active compound from the SAR study of brassinosteroid cytotoxicity on cancer cells was 9. Changing 6-oxo-7-oxalactone to 6-oxo functionality dramatically increases the brassinosteroid growth inhibitory activity. Thus, 9 gave about 3-times stronger response then 28-homobrassinolide 2. The more flexible conformational behavior of the 24R side chain in 24-epicastasterone compared with castasterone, as determined by NMR and modeling studies, is a critical property decreasing its anticancer activity. The 24R side chain is also a decisive group lowering cytotoxicity of BRs. An ethyl group in the side chain at C24 in 9 and 2 are somewhat more effective then corresponding analogues with C24 methyl. At least the zero growth inhibitory activity of β-ecdysone containing 2β,3β,22α-functionality indicates a high probability that 3α-hydroxy group, 2α,3α-vicinal diol or 3α,4α-vicinal diol may be important for the brassinosteroid anticancer activity.

### Example 2

### Effect of Novel Compounds on Breast and Prostate Cancer Cells

The most promising and easily available brassinosteroid analogues with interesting anticancer properties we selected for the next experiment on MCF-7 (estrogen receptor-α-positive) and MDA-MB-468 (estrogen receptor-α-negative) breast cancer cell lines and/or LNCaP (androgen-sensitive) and DU-145 (androgen-insensitive) prostate cancer cell lines.

Prostate cancer in humans is however very complex as they progress from an androgen-responsive to an androgen-unresponsive state, and by the clinical diagnosis, most prostate cancers represent a mixture of androgen-dependent versus androgen-independent cells. Whereas androgen-sensitive cells undergo rapid apoptosis on androgen ablation, androgen-insensitive cells by-pass the apoptosis pathways during androgen withdrawal, although they retain the molecular machinery for apoptosis. Mortality from prostate cancer generally occurs from the proliferation and invasion of these androgen-unresponsive cells, which fails to undergo apoptosis culminating into hormone-refractory prostate cancer for which no cure but only palliative treatment is available. There are also similar patterns in hormone responsibility of estrogen-sensitive or estrogen-insensitive breast cancers. Therefore, eliminating both cell types of the cancers seems to be an intensifying approach for treatment of this disease.

Cell viability in this case was assessed by 3-(4,5-dimethylthiozol-2-yl)-2,5-diphenyltetrazolium bromide (MTT) to determine IC₅₀ concentrations of studied agents as described previously. The cells were seeded at 4.5×10³ (MCF-7, DU-145) or 5×10³ (MDA-MB-468, LNCaP) cells per well in medium containing the steroid-stripped serum to 96-well plates. Cells were grown 24 h (MCF-7, MDA-MB-468, DU-145) or 48 h (LNCaP), respectively. The cells (70% to 80% confluent) were treated with brassinosteroids for 6, 12 and 24 h in cell culture medium. Cells that were used as controls were incubated with the maximum used amount of steroid diluent DMSO only. The concentration leading to 50% inhibition of viability (IC₅₀) after 24 h was determined by measuring MTT reductase activity. The absorbance was read using an ELISA reader Labsystem Multiscan RC at 570 nm. The viability of treated cells was related to the viability of control cells that represented 100% viability. Each experiment was performed in triplicate and independently repeated at least four times.

TABLE 2
IC₅₀ (μmol/L) concentrations of brassinosteroids
9 and 6 were determined by MTT cytotoxicity test.

IC₅₀ (μmol/L)

| Cell line | 9 | 6 |
|---|---|---|
| MCF-7 | 40 ± 1.5 | 60 ± 1.8 |
| MDA-MB-468 | 65 ± 2.8 | 68 ± 2.5 |
| LNCaP | 45 ± 2.3 | 60 ± 3.1 |
| DU-145 | 45 ± 2.8 | 65 ± 0.9 |

The results are means ± SD of three independent experiments performed in triplicate.

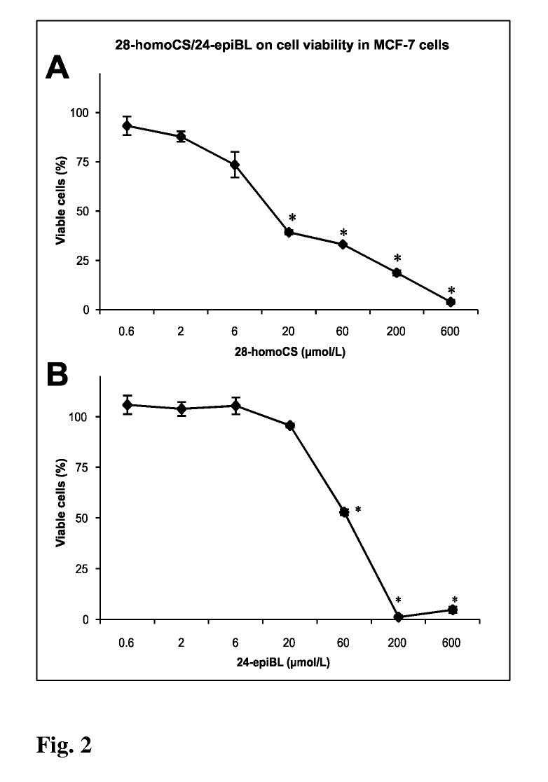

To evaluate the effect of the selected brassinosteroids (9 and 6) on the viability of hormone sensitive and insensitive cancer cell lines (MCF-7, MDA-MB-468, LNCaP and DU-145), we analyzed cell viability after the BR treatment by MTT assay. The cytotoxic concentrations were reached with all of the analogues in the four cancer cell lines. Brassinosteroid 9 inhibited cell growth in a dose-dependent manner in all cancer cell lines as does 6. MCF-7 breast cancer cells were the most sensitive to 9 treatment than the other three cell lines. Brassinosteroid 6 showed the cytotoxic effects at lower concentration tested (FIG. 2). Employing the MTT assay, we also found the concentrations of both brassinosteroids leading to 50% inhibition of cell viability (IC₅₀) after 24 h (Table 2). Interestingly, there was a little difference between the potency of 9 or 6 on different cell lines as IC₅₀ values moved consistently around 43.3±2.4 μmol/L and 61.7±1.7 μmol/L, respectively. There was only one exception in the case of MDA-MB-468 estrogen-insensitive breast cancer cell line as these cells were more resistant to application of both brassinosteroids (9 IC₅₀: 65±2.8 μmol/L, 6 IC₅₀: 68±2.5 μmol/L).

### Example 3

### Cell Proliferation by Bromodeoxyuridine Incorporation Assay

To analyze if the inhibition in cell viability was due to decreased cell proliferation, we measured DNA synthesis in the presence of BRs. The effect of brassinosteroids 9 or 6 on cell proliferation and DNA replication of the cancer cells was measured by BrdU incorporation assay. This immunostaining was based on nuclear incorporation of bromodeoxyuridine (BrdU) into DNA in place of thymidine during the S-phase of cell cycle detected by anti-specific-BrdU antibody following membrane permeabilization as described previously (Gratzner, Science. 1982, 218, 474-475). Cells (60 to 70% confluence) were cultured with brassinosteroids (IC₅₀) for 6, 12 and 24 h in 60-mm culture dishes with coverslips as described above. The BrdU reagent (0.1 mM; Sigma, MO, USA) was added in culture medium with cells for 4 h in a CO₂ incubator at 37° C. After incubation the cells were washed three times with PBS and fixed with cold acetone-methanol (1:1, v/v) for 10 min. The cells were denaturated by HCl (1:5; Lachema, Czech Republic) and incubated with anti-specific-BrdU antibody (diluted 1:100 in PBS; clone Bu20a, DakoCytomation, Denmark) for 60 min in the dark. The cells were then washed three times in PBS (10 mM, pH 7.4) and incubated with goat anti-mouse fluorescein isothiocyanate (FITC)-labeled secondary antibody (Sigma, MO, USA) for 60 min in the dark. The cells were then washed three times in PBS and incubated with DAPI (50 μg/mL; Sigma, MO, USA) for 10 min in the dark. The coverslips with cells were washed in deionized water and mounted on glass slides, using the hydrophilic medium Mowiol (Calbiochem, Calif., USA) in glycerol-PBS (1:3, v/v) for fluorescence. Cells were visualized using microscopy and those incorporating BrdU (S phase cells) were counted in at least 25 fields and compared with the total number of all cells.

TABLE 3
Cell proliferation by BrdU incorporation assay.

| Control/BRs | Cell line | | | |
|---|---|---|---|---|
| (IC₅₀) | MCF-7 | MDA-MB-468 | LNCaP | DU-145 |
| Ctrl (6 h) | 50 ± 3.6 | 64 ± 1.5 | 44 ± 6.0 | 51 ± 3.8 |
| 9 (6) | 46 ± 2.5 | 55 ± 5.0 | 25 ± 3.0* | 41 ± 4.5 |
| 6 (6) | 42 ± 3.2 | 53 ± 4.4* | 36 ± 5.5 | 32 ± 3.8* |
| Ctrl (12 h) | 53 ± 3.5 | 58 ± 3.0 | 52 ± 7.6 | 54 ± 4.2 |
| 9 (12 h) | 31 ± 3.8* | 48 ± 4.6 | 28 ± 4.6* | 36 ± 4.4* |
| 6 (12 h) | 40 ± 7.1 | 55 ± 3.5 | 31 ± 2.0* | 27 ± 5.3* |
| Ctrl (24 h) | 69 ± 4.5 | 64 ± 5.6 | 59 ± 8.0 | 56 ± 6.5 |
| 9 (24 h) | 29 ± 4.0* | 28 ± 4.6* | 27 ± 2.1* | 38 ± 4.0* |
| 6 (24 h) | 36 ± 5.5* | 43 ± 3.6* | 29 ± 4.7* | 16 ± 1.5* |

MCF-7, MDA-MB-468, LNCaP and DU-145 cancer cells treated by brassinosteroids 9 or 6 (IC₅₀; for 6/12/24 h) and compared with untreated control cells (Ctrl). Data indicate percentage (%) of proliferating BrdU positive cells. The results are means ± SD of three independent experiments.
*denotes the value that is significantly different from control value at p < 0.05.

The breast and prostate cancer cell lines were treated by brassinosteroids 9 or 6 in IC₅₀ concentrations for 6, 12, and 24 h and the results compared with the untreated controls. Both brassinosteroids inhibited cell proliferation in a dose and time-dependent manner in all cell lines tested (Table 3). Treatment with brassinosteroids resulted in a decreased percentage of BrdU positive cells. The application of 9 (IC₅₀ for 24 h) led to the most significant decrease in number of proliferating cells in the MCF-7 cell line (from 69±4.5% in controls to 29±4.0% in 9) (Table 3). These data indicate that brassinosteroids are able to reduce growth of several breast and prostate cancer cell lines. It is yet unclear whether the hormone-responsive cell lines are more sensitive and whether the cytotoxic effects of brassinosteroids are mediated by brassinosteroid-steroid receptor interaction. The study of the correlation between estrogen receptor (ER) status of the tumor and a positive response to endocrine therapy, would leed to the development of estrogen antagonists.

### Example 4

### Brassinosteroids Regulate Cell Cycle

In the MCF-7 breast cancer cell line that has been the most widely studied experimental cell system, the typical growth inhibitory response to antiestrogens is decrease in the proportion of cells synthesising DNA (S phase) after antiestrogen treatment. This decrease in S phase coincides with an increase in the proportion of cells in G₀/G₁ phase. The activity of the androgen receptor (AR) is also regulated by alterations in cell signaling. There has been reported ligand independent activation of AR in prostate cancer cells by different growth factors. As the AR can be activated in the absence of hormone, it appears that the conditions may be more restricted than those observed for ER. Use of physiologic doses of ligand (dihydrotestosterone, DHT) is of importance as the LNCaP cells proliferate only at DHT concentration of 10⁻⁹ M or lower. Higher doses of ligand actually inhibit proliferation. In contrast to proliferation, transactivation of AR specific target genes (e.g. PSA) continues to increase with DHT doses of 10⁻¹⁰ M and higher. It might be accepted for estrogen or androgen receptors as stronger cytotoxic activities of brassinosteroids 9 or 6 on hormone-sensitive (MCF-7, LNCaP) compared to hormone-insensitive (MDA-MB-468, DU-145) cell lines were found. Therefore we explored the possibility of cell cycle perturbation by BRs in different cancer cell lines. Flow cytometry was used to evaluate the number of cells in the particular phases of the cell cycle, including subG₁ peak detection. Control and treated cells were washed twice with cold PBS and centrifuged at 360×g for 10 min at 4° C., and fixed with chilled ethanol (70%; v/v) by low-speeded vortexing. For detection of DNA content analysis, propidium iodide staining was used. The cells were analyzed using a FACSCalibur flow cytometer (BD Biosciences, San Jose, Calif.).

TABLE 4
Cell cycle distribution of MCF-7, MDA-MB-468, LNCaP,
and DU-145 cells by flow cytometry analysis.

| | Control/BRs | Apoptosis | Cell cycle distribution | | |
|---|---|---|---|---|---|
| Cell line | (IC₅₀; 24 h) | subG₁ | G₁ | S | G₂/M |
| MCF-7 | Ctrl | 17% | 60% | 14% | 26% |
| | 9 | 15% | 80% | 11% | 9% |
| | 6 | 13% | 81% | 7% | 12% |
| MDA-MB-468 | Ctrl | 10% | 52% | 31% | 17% |
| | 9 | 35% | 74% | 17% | 9% |
| | 6 | 75% | 88% | 10% | 2% |
| LNCaP | Ctrl | 8% | 70% | 14% | 16% |
| | 9 | 18% | 94% | 2% | 4% |
| | 6 | 47% | 86% | 5% | 9% |
| DU-145 | Ctrl | 20% | 62% | 15% | 23% |
| | 9 | 28% | 46% | 8% | 46% |
| | 6 | 24% | 53% | 8% | 39% |

Histograms of the brassinosteroids treated cells were compared with control untreated cells histograms. The percentages indicate number of cells in sub G₁ fraction and G₁, S, G₂/M phases of the cell cycle.

The flow cytometry analysis showed a block in G₁ phase of the cell cycle in MCF-7, MDA-MB-468 and LNCaP cell lines after treatment with brassinosteroids 9 or 6 (Table 4). Brassinosteroids caused a decrease in percentage of all cell lines in S phase of the cell cycle. In DU-145 cells, however, an increase in cell percentage in the G₂/M phase and a reduction of cells in the other cell cycle phases after brassinosteroid treatments have been observed (Table 4).

### Example 5

### Effect of Brassinosteroids 9 and 6 on Expression of Cell Cycle Proteins

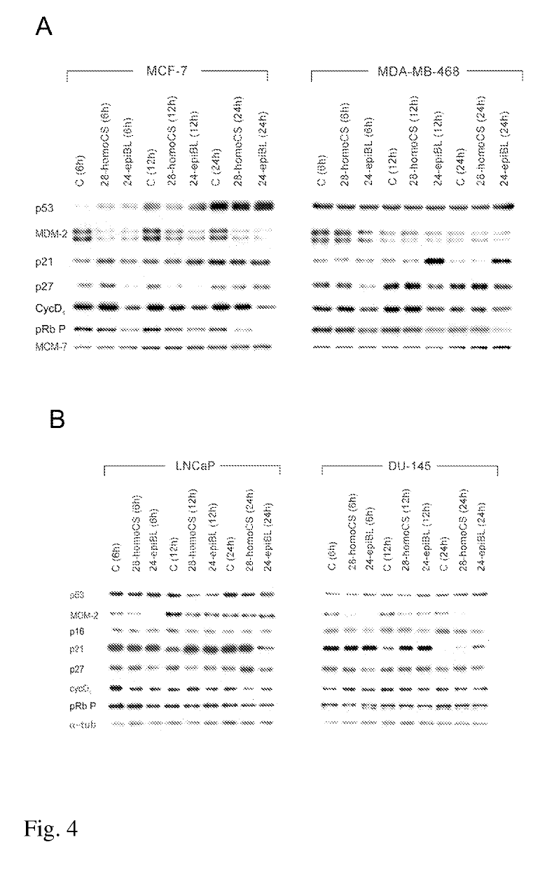

We examined the effect of brassinosteroids 9 or 6 on the cyclin-dependent kinase (cdk) inhibitors p21^{Waf1/Cip1} and p27^{Kip1} that are key regulators of cell cycle progression and function by inhibiting the cyclin/cdk activities. In MCF-7 cells, treatment by 9 led to increase in p21^{Waf1/Cip1} and p27^{Kip1} expression after 6 h, whereas in MDA-MB-468 no significant changes were found. After 24-epiBL treatment of MCF-7 cells, expression of p21^{Waf1/Cip1} was unchanged, expression of p27^{Kip1} resulted in decrease after 6 and 12 h exposition of 6. In MDA-MB-468 cells, the level of cdk inhibitor protein p21^{Waf1/Cip1} was significantly increased after 12 and 24 h treatment with 24-epiBL (FIG. 4A). In contrast, p27^{Kip1} protein expression was decreased in all time points in these cells. In LNCaP cells, we found decreased level of p21^{Waf1/Cip1} after 12 h and increased level of p27^{Kip1} after 24 h after 9 or 6 treatment led to decreased expression of p21^{Waf1/Cip1} after 6 and 24 h treatment and p27^{Kip1} protein expression after 6 and 12 h in LNCaP cells. Both brassinosteroids resulted in increase of p21^{Waf1/Cip1} and p27^{Kip1} expression after 12 and 24 h in DU-145 cells (FIG. 4B).

In MCF-7 cells, after 6 h exposition to brassinosteroids 9 or 6 a slight increase in p53 protein expression attended by decreased expression of MDM-2 regulator of p53 degradation after all time points was seen. In MDA-MB-468 cells, the protein levels of p53 and MDM-2 remained unchanged after treatment with both types of BRs (FIG. 4A). In LNCaP cells, decreased expression of p53 after brassinosteroids 9 or 6 treatment was found after 12 and 24 h, whereas in DU-145 no p53 expression changes were found. The expression of MDM-2 was increased after 9 treatment (6 h) and unchanged after 6 treatment in LNCaP cells. DU-145 cells treated by brassinosteroids 9 or 6 of resulted in decreased level of MDM-2 in all time points (FIG. 4B).

Treatment by 24-epiBL (6/12/24 h) of both breast cancer cells led to decreased expression of cyclin D₁. The expression of both phosphorylated and dephosphorylated forms of the retinoblastoma protein (Rb) decreased after application of brassinosteroids 9 or 6 in MCF-7 and MDA-MB-468 cells (FIG. 4A). In LNCaP cells treated by both BRs the level of cyclin D₁ was reduced, whereas in DU-145 the level of cyclin D₁ remained uneffected. The expression of Rb protein was slightly decreased after treatment with BRs in LNCaP cells. In DU-145 no changes were found (FIG. 4B).

### Example 6

### Detection of Apoptosis in Cancer Cells

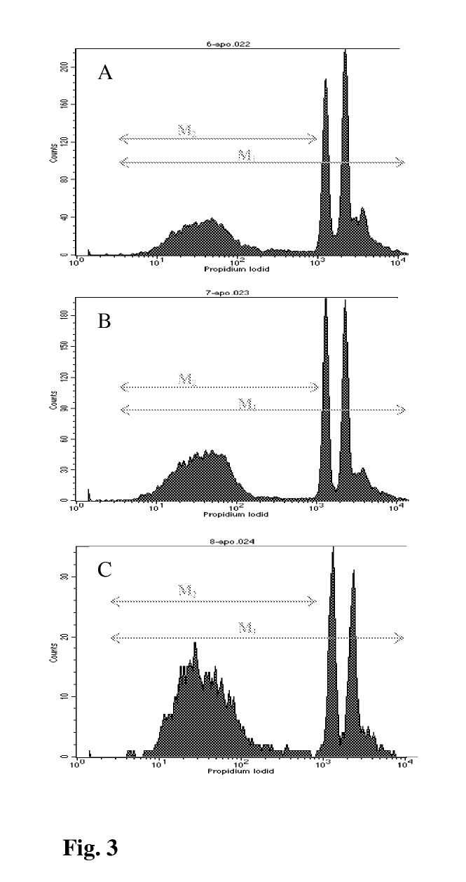

Flow-cytometric detection of apoptosis. Several studies have declared that the induction of apoptosis may be cell cycle-dependent. Therefore, in our next series of experiments, we tested if brassinosteroids 9 or 6 cause apoptosis of the breast and prostate cancer cells via cell cycle blockage. From this reason we carried out DNA cell cycle analysis by flow cytometry based on detection of endonucleolytic DNA degradation that results in extraction of low molecular weight DNA from the cells. Flow cytometry, used as a conventional technique for detection of a particular form of cell death, showed an appearance of a subG₁ peak in the hypodiploid region of the cell cycle-related DNA histograms. The analysis demonstrated that BRs increased the proportion of the subG₁ fraction in brassinosteroid treated MDA-MB-468 (FIG. 3), LNCaP and slightly in the DU-145 cells in comparison with the untreated controls (Table 4). The subG₁ peak containing apoptotic bodies was however not observed in MCF-7 cells. The observation of apoptosis after brassinosteroid treatment is important because the molecular analyses of human cancers have revealed that cell cycle regulators are frequently mutated in most common malignancies.

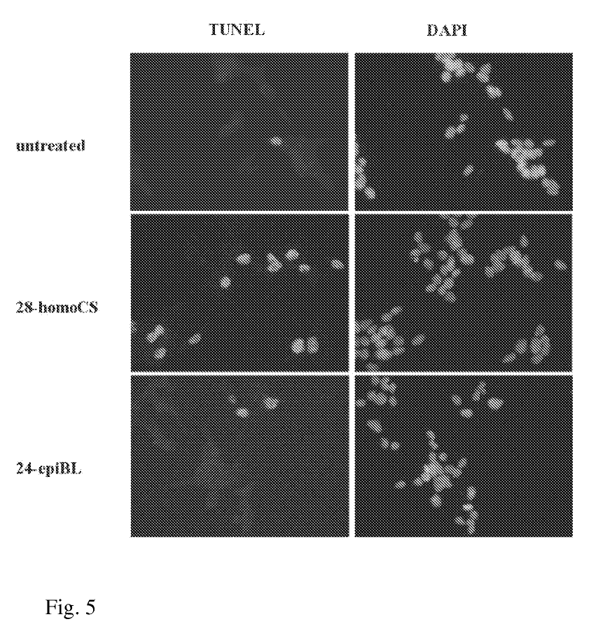

TdT-Mediated dUTP nick end labeling (TUNEL) detection of apoptosis index. For detection of apoptotic cells, the TUNEL assay was used. Cells were cultured with brassinosteroids 9 or 6 (IC₅₀) for 6/12/24 h in 60-mm culture dishes with coverslips and treated as described above. After period of treatment, the cells were washed with PBS and fixed on the slides with cold acetone-methanol (1:1, v/v) for 10 min. Apoptosis-induced nuclear DNA fragmentation was detected by terminal deoxynucleotidyl transferase-mediated UTP nick end labeling (TUNEL) technique according to the producer protocol (In Situ Cell Death Detection Kit; Roche Diagnostics, Mannheim, Germany). The cells were then washed three times in PBS and stained with 4′-6-diamidino-2-phenylindole (DAPI; 50 μg/ml; Sigma, St. Louis, Mo., USA) for 10 min in the dark. The coverslips with cells were washed in deionized water and mounted on glass slides, using the hydrophilic medium Mowiol (Calbiochem, Fremont, Calif., USA) in glycerol-PBS (1:3, v/v) for fluorescence. Cells were visualized using fluorescence microscopy and compared with control cells. TUNEL staining achieved to confirm apoptosis in all cell lines. Cell treatment with brassinosteroids in all time points (IC₅₀; 6/12/24 h) slightly but not very significantly increased the number of TUNEL positive cells (Tab. 5). Important increase of percentage of apoptotic, TUNEL positive, cells by 25.8% after treatment with 9 (IC₅₀ for 24 h) and by 21.9% after treatment with 6 (IC₅₀ for 24 h) was found in LNCaP cells. The effects of compounds 9 and 6 on LNCaP cells are presented in FIG. 5.

TABLE 5
Detection of DNA strand breaks in apoptotic
cell nuclei by TUNEL method.

| Control/BRs | Cell line | | | |
|---|---|---|---|---|
| (IC₅₀) | MCF-7 | MDA-MB-468 | LNCaP | DU-145 |
| Ctrl (6 h) | 0.0 ± 0.0 | 0.0 ± 0.0 | 0.3 ± 0.6 | 0.0 ± 0.0 |
| 9 (6 h) | 10.6 ± 1.5* | 1.7 ± 0.6 | 7.3 ± 2.1* | 6.0 ± 2.0* |
| 6 (6 h) | 2.7 ± 1.2* | 4.7 ± 0.6* | 10.7 ± 4.0* | 4.7 ± 2.5* |
| Ctrl (12 h) | 0.0 ± 0.0 | 0.0 ± 0.0 | 0.6 ± 0.6 | 0.0 ± 0.0 |
| 9 (12 h) | 13.7 ± 3.8* | 2.3 ± 0.6 | 15.7 ± 2.5* | 7.5 ± 4.0* |
| 6 (12 h) | 8.3 ± 3.1* | 16.7 ± 4.7* | 12.2 ± 5.1* | 5.6 ± 2.0* |
| Ctrl (24 h) | 0.3 ± 0.6 | 0.0 ± 0.0 | 0.6 ± 0.6 | 0.0 ± 0.0 |
| 9 (24 h) | 13.7 ± 2.6* | 4.3 ± 0.6* | 25.8 ± 2.5* | 8.0 ± 4.0* |
| 6 (24 h) | 11.7 ± 3.8* | 18.3 ± 3.0* | 21.9 ± 3.0* | 5.1 ± 5.6* |

MCF-7, MDA-MB-468, LNCaP and DU-145 cells were treated with compounds 9 or 6 for 6/12/24 h in IC₅₀ concentration and detected TUNEL method in comparison with untreated control cells. The cells were washed with PBS and fixed on the slides with cold acetone-methanol (1:1 v/v) as described above in Materials and Methods. Ctrl (6 h), Ctrl (12 h), Ctrl (24 h)-untreated controls; 9 (6 h), 9 (12 h), 9 (24 h) - cells trested with compound 9; 6 (6 h), 6 (12 h), 6 (24 h) - cells trested with compound 6. Data indicate percentage (%) of TUNEL positive cells. The results are means ± SD of three independent experiments.
*denotes the value that is significantly different from control value at p < 0.05.

### Example 7

### Western Blot Analysis of Pro- and Anti-Apoptotic Proteins

The cells were seeded in a density 1.6×10⁴ cells/cm² (LNCaP), 1.4×10⁴ cells/cm² (DU-145, MDA-MB-468 and MCF-7) using culture medium in 100-mm culture Petri dishes. After reaching of 70-80% confluence, the cells were treated with tested compounds 9 in concentration IC₅₀ that were evaluated by MTT analysis described above. DMSO was used as a vehicle for controls. After 6, 12 and 24 h treatment, the cells were washed with cold PBS and scraped in ice-cold protein extract ion buffer (50 mM HEPES, pH 7.5; 150 mM NaCl; 1 mM EDTA; 2.5 mM EGTA; 10% glycerol; 0.1% Tween 20) with protease and phosphatase inhibitors (25 μL/mL phenylmethanesulphonyl fluoride; 2.5 μL/mL leupeptin; 0.1 mM Na₃VO₄; 2.5 μL/mL aprotinin; 10 mM β-glycerol-phosphate; 1 mM dithiothreitol). The lysates were collected into microfuge tube and incubated on ice for 1 h. Cells were incubated 60 min at 4° C. under time by time shaking in protein extraction buffer. After centrifugation at 45 000×g for 30 min at 4° C., supernatant was collected, aliquoted, and stored at −80° C. The protein content in the lysates was measured by a Bredford assay (Bio-Rad Laboratories, Hercules, Calif., USA) according to the manufacture's protocol.

For Western blot analysis, equal aliquots of proteins (15-30 μg/well) were loaded on 10% or 12% SDS-PAGE gels and transferred onto nitrocellulose membranes (Amersham Biosciences, Vienna, Austria) by semi-dry electrophoretic transfer. Coloured marker (Rainbow™, Amersham Biosciences, Vienna, Austria) was used as a protein molecular weight standard. The non-specific binding sites were blocked by incubating the blot with 5% (w/v) non-fat dry milk in PBS for 2 h. The blot was incubated overnight in 4° C. with appropriate primary antibody that detects the protein epitope of interest and finally washed in PBS with 0.1% Tween 20 for 1 h. The blot was incubated with the diluted secondary goat anti-mouse IgG-horseradish peroxidase conjugated antibody (dilution 1:6000, Santa Cruz Biotechnology, Santa Cruz, Calif., USA) or goat anti-rabbit IgG-horseradish peroxidase conjugated antibody (dilution 1:2000, DakoCytomation, Glostrup, Denmark) for 45 min in 4° C. The membrane was washed in PBS with 0.1% Tween 20 for 1 h. The proteins were detected using a chemiluminescence detection system (Amersham Biosciences, Vienna, Austria) according to the protocol provided by the manufacturer. Equality of loaded proteins was confirmed by Ponceau S membrane staining (Sigma, St. Louis, Mo., USA) and by detection of anti-tubulin-α or anti-mcm-7 antibodies. The experiments were repeated three times. The protein expressions in treated cells were compared to untreated controls.

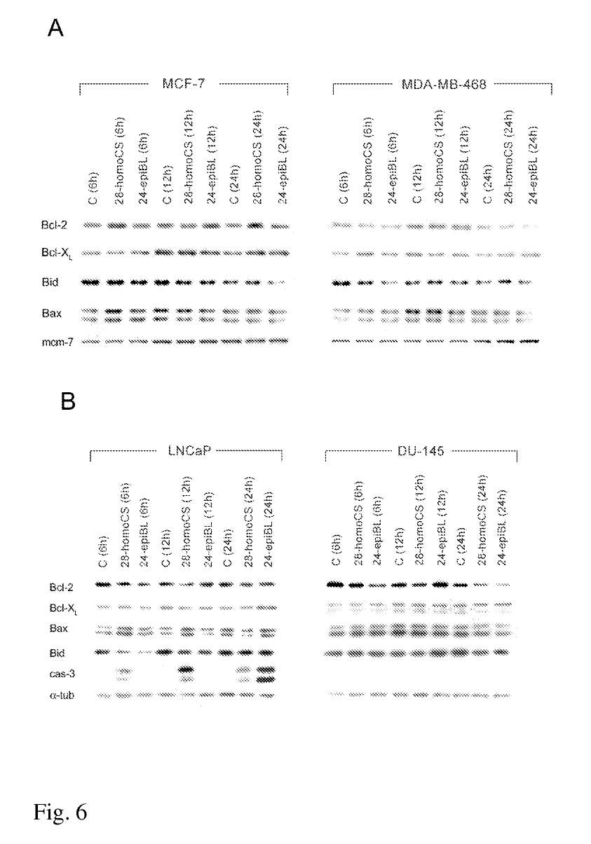

Western blot analysis was used to detect changes in apoptosis related protein expression in breast and prostate cancer cell lines. To monitor changes over the 24 h treatment, we collected the cells after 6, 12 and 24 h treatment with BRs in concentration IC₅₀ evaluated by MTT assay. Changes in apoptosis related protein expression after treatment with BRs are shown in FIG. 6. In MCF-7 and MDA-MB-468 breast cancer cell lines, Western blot analysis showed no significant changes in expression of apoptosis related proteins after treatment by tested BRs (FIG. 6A). Expression of anti-apoptotic protein Bcl-2 decreased after treatment with 9 (6, 12 and 24 h) or 6 (6 and 24 h) in LNCaP and DU-145 cells, whereas expression of Bcl-X_{L} protein was unchanged after exposure to both BRs at all time points (FIG. 6B). LNCaP cells showed an increased level of pro-apoptotic Bax after the 9 treatment after 6 and 12 h and decreased expression of pro-apoptotic uncleaved protein Bid after 6 and 12 h treatment with both BRs. This decreased expression of Bid can indicate a cleavage of the protein after treatment by BRs. In DU-145 cell line, expression of pro-apoptotic proteins Bax and Bid was unchanged. Brassinosteroid 9 treatment of LNCaP cells induced degradation of caspase-3 into its cleaved fragments after all time points (6, 12 and 24 h). Brassinosteroid 6 resulted in an activation of caspase-3 after 24 h treatment of LNCaP cells. On the other hand, caspase cleavage was not found in DU-145 cells. Poly-(ADP-ribose) polymerase (PARP) positive expression in controls of prostate cancer cell lines was observed but brassinosteroids had no effect on its expression nor on its degradation within the period 6/12/24 h (FIG. 6B).

In this study we have shown that compound 9 treatment to LNCaP resulted in significant decrease in the levels of anti-apoptotic Bcl-2 protein and increase in the pro-apoptotic Bax protein. Moreover, a degradation of procaspase-3 into cleaved fragments was detected after all time points of treatment with 9 and after 24 h treatment with 6 in LNCaP cells. This findings whose to iniciation apoptosis changes in LNCaP cells. It has been known that the execution mechanism of apoptosis is mediated by caspase cascade activation (Budihardjo et al., Annu. Rev. Cell Dev. Biol. 15, 269-290, 1999). Caspase-3 is an executioner protease that results in the cleavage of PARP and subsequent DNA degradation and apoptotic death (Allen et al., Cell. Mol. Life Sci. 54, 427-445, 1998; Cain et al., Biochimie 84, 203-214, 2002). These results confirm that brassinosteroids 9 and 6 can support apoptosis with caspase-3 activation and modulations in Bcl-2 family proteins in cell lines derivated from prostate carcinoma.

In breast cancer cell lines, Western blot analysis showed no significant changes in expression of Bcl-2 family protein nor caspase-3 activation after treatment with brassinosteroid 9 and 6.

### Example 8

### Dry Capsules

5000 capsules, each of which contain 0.25 g of one of the compounds of the general formula I mentioned in the preceding Examples as active ingredient, are prepared as follows:

### Composition

| | |
|---|---|
| Active ingredient | 1250 g |
| Talc | 180 g |
| Wheat starch | 120 g |
| Magnesium stearate | 80 g |
| Lactose | 20 g |

Preparation process: The powdered substances mentioned are pressed through a sieve of mesh width 0.6 mm. Portions of 0.33 g of the mixture are transferred to gelatine capsules with the aid of a capsule-filling machine.

### Example 9

### Soft Capsules

5000 soft gelatine capsules, each of which contain 0.05 g of one of the compounds of the formula I mentioned in the preceding Examples as active ingredient, are prepared as follows:

### Composition

| | | |
|---|---|---|
| Active ingredient | 250 | g |
| Lauroglycol | 2 | litres |

Preparation process: The powdered active ingredient is suspended in Lauroglykol® (propylene glycol laurate, Gattefossé S.A., Saint Priest, France) and ground in a wet-pulveriser to a particle size of about 1 to 3 μm. Portions of in each case 0.419 g of the mixture are then transferred to soft gelatine capsules by means of a capsule-filling machine.

### Example 10

### Soft Capsules

5000 soft gelatine capsules, each of which contain 0.05 g of one of the compounds of the formula I, II or III mentioned in the preceding Examples as active ingredient, are prepared as follows:

### Composition

| | | |
|---|---|---|
| Active ingredient | 250 | g |
| PEG 400 | 1 | litre |
| Tween 80 | 1 | litre |

Preparation process: The powdered active ingredient is suspended in PEG 400 (polyethylene glycol of Mr between 380 and about 420, Sigma, Fluka, Aldrich, USA) and Tween® 80 (polyoxyethylene sorbitan monolaurate, Atlas Chem. Inc., Inc., USA, supplied by Sigma, Fluka, Aldrich, USA) and ground in a wet-pulveriser to a particle size of about 1 to 3 μm. Portions of in each case 0.43 g of the mixture are then transferred to soft gelatine capsules by means of a capsule-filling machine.

1. Natural brassinosteroid of the general formula I 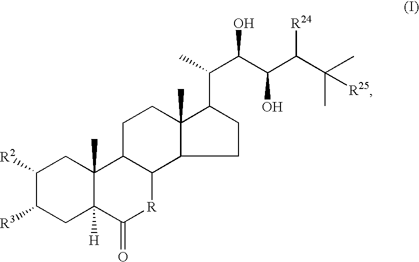
wherein
R is CH₂ or O—CH₂ group,
R² is hydrogen or hydroxyl,
R³ is hydroxyl,
R²⁴ is alkyl or alkenyl, which are selected from the group consisting of methyl, ethyl, propyl, isopropyl, methylen, ethylen and propylen, and
R²⁵ is alkyl selected from the group consisting of methyl and ethyl, and a pharmaceutically acceptable salt thereof,
for use for treating hyperproliferation, treating proliferative diseases and reducing adverse effects of steroid dysfunction in mammals.

2. Natural brassinosteroids of the general formula I according to claim 1 for use for treating of proliferative diseases of mammals characterised by administration of an therapeutically effective amount of natural brassinosteroids to the mammal's hyperproliferating cells in need of such treatment, wherein the natural brassinosteroid derivative is selected from the group consisting of the following compounds: castasterone, 28-homocastasterone, 24-epibrassinolide, dolichosterone, 2-deoxycastasterone, typhasterol, teasterone, 3-oxoteasterone, cathasterone, 6-deoxotyphasterol, 3-dehydro-6-deoxoteasterone, homotyphasterol, homoteasterone, homodolichosterone, 25-methylcastasterone, 25-methyldolichosterone, 2-deoxy-25-methyldolichosterone, and 3-epi-2-deoxy-25-methyldolichosterone.

3. Natural brassinosteroids according to claim 1, wherein R², R³, R²⁴, and R²⁵ have independently at each occurrence (R) or (S) configuration.

4. Natural brassinosteroids for use for inhibiting cell proliferation in mammals comprising administration of a therapeutically effective amount of compound according to claim 1 or a pharmaceutically acceptable salt thereof together with a pharmaceutical carrier.

5. Natural brassinosteroids of the general formula I according to claim 1 for use as growth regulators in animal and human tissue cultures for regulation of proliferation and morphogenesis comprising administering an effective amount of the compound of a pharmaceutically acceptable salt thereof to an animal or human tissue culture.

6. Natural brassinosteroids of the general formula I according to claim 1 for use for inhibiting cell proliferation in mammals comprising administration of a therapeutically effective amount of the compound or a pharmaceutically acceptable salt thereof together in combination with usually used cytostatics, such as mitoxantrone, cis-platinum, methotrexate, taxol, or doxorubicin.

7. Natural brassinosteroids of the general formula I according to claim 1 for use for inducing apoptosis in mammalian cells comprising administering to a subject a therapeutically effective amount of natural brassinosteroid of the general formula I, or a pharmaceutically acceptable salt thereof together with a pharmaceutical acceptable carrier.

8. Natural brassinosteroids of the general formula I for use for treating cancer in mammals comprising administration of a therapeutically effective amount of a compound according to claim 1 or a pharmaceutically acceptable salt thereof to a subject together with a pharmaceutical carrier.

9. Natural brassinosteroids of the general formula I for use for inhibiting of cancer according to claim 8, wherein cancer is of prostate, endometrium or breast origin.

10. Natural brassinosteroids of the general formula I according to claim 1 for use for regulation of adverse effects of steroid disfunctions in mammals cells comprising administration of a therapeutically effective amount of the compound or a pharmaceutically acceptable salt thereof together with a pharmaceutical carrier, wherein the steroid disfunction is osteoporesis, cholesterol metabolism defects, Alzheimer disease, Huntington disease, steroid-induced cataracta, deficiency of P450 oxidoreductase, men infertility and disorder of sexual behaviour and differentiation.

11. Pharmaceutical composition comprising an effective amount of natural brassinosteroids of the general formula I according to claim 1.

12. Pharmaceutical composition according to the claim 14 for use for treatment of mammalian cells and human beings.
